(19) United States
(12) Patent Application Publication
(10) Pub. No.: US 2001/0049351 A1
(43) Pub. Date: Dec. 6, 2001

(54) METHOD OF DETERMINING DOWN-REGULATION OF THE EXPRESSION OF HIV CORECEPTOR, CCR5 WITH PRODUCT R

(76) Inventors: SHALOM Z. HIRSCHMAN, RIVERDALE, NY (US); CHAOWUAN CHEN, EDISON, NJ (US)

Correspondence Address: MYRON COHEN, COHEN PONTANI LIEBERMAN & PAVANE, 551 FIFTH AVE, SUITE 1210, NEW YORK, NY, 10176

(21) Appl. No.: 09/257,739

(22) Filed: Feb. 25, 1999

(51) Int. Cl. A01N 37/18; A61K 38/00

(52) U.S. Cl. 514/2

(57) ABSTRACT

The down-regulation of the expression of the HIV coreceptor, CCR5 in H9 lymphoma or U937 promonocyte cells by Product R, a peptide-nucleotide preparation, is determined by a method comprising the steps of cell culture, electroporation, reverse transcription-polymerase chain reaction and electrophoresis.

### BACKGROUND OF THE INVENTION

1. Field of the Invention

The present invention relates to a method of determining the level of the expression of human immunodeficiency virus (HIV) coreceptor, CCR5. Specifically, the method demonstrates that CCR5 is down regulated by Product R, a peptide-nucleic acid preparation, in H9 T lymphoma and U937 promonocyte cells. The invention may serve as a functional assay for the biological activity of Product R.

2. Description of the Related Art

Chemokines or chemotactic cytokines possess the property of both chemoattractants and cytokines. So far about 50 heterogeneous proteins have been identified, and their biological sources are mainly stromal cells, muscle cells, endothelial and epithelial cells, monocytes and T lymphocytes. A spectrum of biological activities of chemokines has been demonstrated in vitro and in vivo, including regulation of the migration and activation of leukocytes, enhancement of immunoglobulin production by B cells, degranulation of NK cells and cytotoxic T cells, and blockade of the entry of HIV into macrophages and T cells.

Several lines of experimental evidence delineate the critical role of chemokines and chemokine receptors in HIV infection of human cells. First, most HIV strains or clinical HIV isolates cannot effectively infect human cells only expressing the CD4 molecule or the primary HIV receptor, suggesting other coreceptors are involved in control of the entry of the virus. In most cases, a proximal interaction of chemokine receptors and the CD4 molecule is required for HIV infection of human cells. Secondly, the presence of high levels of certain chemokines correlates to resistance to HIV infection or slow progress of the clinical course of AIDS. The binding of certain chemokines to their receptors expressed on the surface of macrophages and T lymphocytes block the entry of HIV. Thirdly, high percentages of individuals who are resistant to HIV infection carry a homozygous 32-bp deletion (A32 allele) in one of the chemokine receptors, namely CCR5. A slow clinical course of HIV infection has been observed in those individuals having heterozygous for the A32 allele.

Among all chemokine receptors identified so far, CCR5 and CXCR4 have been considered as two major coreceptors for HIV entry of target cells. CCR5 on macrophages, monocytes and primary T cells is a primary coreceptor for macrophagetropic HIV strains, while CXCR4 on T cell lines and primary T cells is for T-tropic HIV strains. CCR5 and CXCR4 expressed on primary T cells serve as coreceptors for dualtropic HIV strains. Furthermore, CCR5 appears to be used by viruses from patients in early stages of HIV infection, whereas CXCR4 seems to dominate in later stages of AIDS, which is significantly associated with rapid decline of CD4⁺ T cells, tropism change and opportunistic infections. In several experimental systems, it has been demonstrated that SDF-1 blocks the entry of T cell-line tropic HIV and that RANTES, MIP1-α and MIP-1β are inhibitors of macrophage-tropic HIV-1. Although it remains unclear how these chemokines achieve their HIV-suppressive effects at the molecular level, it is generally accepted that proximal interaction of the CD4 molecule with chemokine receptors is required for most HIV strains to enter the target cells. Recent studies suggest that a complex of CD4 and gp120 interacts with CCR5 and this interaction is inhibited by beta-chemokines or neutralizing antibodies to gp120. The CCR5 binding domain on gp 120 is defined as a minimal region containing the CD4 binding site, overlapping epitopes and the V3 region.

Due to the critical role of the interactions of CD4, gp120 and chemokine receptors in the course of HIV entry, efforts have been made to develop antagonistic agents for those molecules, including soluble gp41 DP178, soluble CD4 and N-terminally-truncated RANTES (a CCR5 blocker without chemotactic activity). These antagonists demonstrated promising HIV-blocking activities in vitro and/or in vivo, and the CCR5 blocker simply mimics the protective effect of the CCR5 deletion. From a clinical point of view, molecular targeting of CCR5 seems to have advantages over that of CXCR4 in treatment of HIV infection, because 1) CCR5 is a primary coreceptor for macrophage-tropic HIV-1 strains, which represent a majority of clinical HIV isolates, 2) CCR5 is a major HIV coreceptor expressed predominantly in early stages of HIV infection, and 3) blocking or deletion of CCR5 would not cause any side effects and dysfunction of the immune system.

Product R (RETICULOSE) is a synthesized preparation that contains a mixture of peptide nucleic acids, breakdown components of bovine serum albumin, and, probably, free nucleosides. Although little is known about the chemical nature of Product R, its biological activities have been demonstrated in effective treatment of influenza in the 1930's, in stimulation of growth of bone marrow cells and granulocytes in rabbits post-irradiation in the 1950's, and significant improvement of life quality and immunologic profile of AIDS patients in a most recent clinical trial. To understand Product R-mediated immunoregulatory activities, Chen and Hirschman have made efforts in testing its biological effect on the production of cytokines by HI V-infected human T cell lines and primary peripheral blood mononuclear cells (PBMCs). J. Investig. Med 1996; 44:347-351. Their primary finding clearly demonstrated that Reticulose potentiated the production of IFN-gamma and IL-6 as well as inhibited HIV replication in PBMCs.

Elucidation of interaction of chemokines and their receptors has been shown to be important in understanding of pathogenesis of HIV infection and evaluation of molecular mechanisms of anti-viral agents. It would be of great interest to test immunoregulatory activities of Product R in this aspect based on the following hypotheses or rationale. 1) The components of Product R may directly interact with immune cells (e.g. macrophages, dendritic cells, monocytes, CD4⁺ and CD8⁺ T lymphocytes) on the cell surface and/or intracellularly at the receptor level or at the RNA or DNA level. 2) The binding of Product R to the receptors for HIV may directly interfere with the interaction of the V3 domain of HIV gp120 with its receptors such as CD4 and chemokine receptors, resulting in a blockade of the entry of HIV into the target cells. 3) Product R may down-regulate the expression of the viral receptor (CD4 molecule) and coreceptors (chemokine receptors), causing a physical or functional reduction of viral entry. Alternatively, the binding of the components of Product R to the viral receptors may trigger the process of endocytosis of the receptors and subsequently lead to a significant decrease in availability of the viral receptors. Since the interaction of peptide nucleic acids with cellular mRNA/DNA may cause recombination or point mutation in the targeted cellular nucleotide sequence, it is conceivable that Product R may down-regulate the expression of viral receptors in a similar manner. Therefore, if any mechanisms mentioned above are operative, Product R is capable of converting HIV-susceptible target cells into HIV-resistant cells.

### SUMMARY OF THE INVENTION

Accordingly, an object of the present invention is to develop a method to determine the function of Product R by evaluating the level of the expression of HIV coreceptors, in H9 Tlymphoma and U937 promonocyte cells.

Another object of the present invention is to determine the function of Product R by evaluating the level of the expression of HIV coreceptor CCR5.

The present invention may be used as a biological assay system for the quality control of the Product R preparation.

A further object of the present invention is to reduce the expression of HIV coreceptor CCR5 in a patient by parenterally administering an effective CCR5 reduction amount of Product R to the patient so that HIV infection is treated or prevented.

Other objects and features of the present invention will become apparent from the following detailed description considered in conjunction with the accompanying drawings. It is to be understood, however, that the drawings are designed solely for purposes of illustration and not as a definition of the limits of the invention, for which reference should be made to the appended claims.

### BRIEF DESCRIPTION OF THE DRAWINGS

In the drawings:

FIG. 1 shows a dose-responsive reduction of the expression of HIV corecepotor CCR5, in H9 lymophoma cells by Product R.

FIG. 2 shows a dose-responsive reduction of the expression of HIV coreceptor CCR5 in U937 cells by Product R.

### DETAILED DESCRIPTION OF THE PRESENTLY PREFERRED EMBODIMENTS

Preparation of Product R: as used herein, Product R is the product produced according to either of the following methods.

### Method I For Preparing Product R

Suspend about 35.0 g of casein, about 17.1 g of beef peptone, about 22.0 g of nucleic acid (RNA), about 3.25 g bovine serum albumin in about 2.5 liters of water for injection USP at about 3 to 7° C. in a suitable container and gently stir until all the ingredients have been properly wet. Carefully add while stirring about 16.5 g of sodium hydroxide (reagent grade ACS) and continue stirring until sodium hydroxide completely dissolved. Autoclave at about 9 lbs pressure and 200-230° F. for a period of time until RNA is completely digested, for example, about 4 hours. At the end of the period, the autoclave is stopped and the reaction flask and contents are permitted to slowly cool to ambient temperature. Then cool for at least six hours at about 3-8° C. The resulting solution is filtered through 2 micron and 0.45 micron filters using inert gas such as nitrogen or argon at low pressure (1-6 psi). In a similar manner the solution is filtered again through 0.2 micron pyrogen retention filters. The resulting filtrate is sampled and assayed for total nitrogen. A calculation is then performed to determine the quantity of cooled water for injection to be added to the filtrate to yield a diluted filtrate with a nitrogen content between about 165-210 mg/ml, the final volume is approximately 5 liters. The pH is then adjusted with either concentrated HCl (reagent grade ACS) or 1.0 normal Noah to about 7.3-7.6 range. The diluted solution is then filtered again through 0.2 micron filters with inert gas at low pressure. The final filtrate is then filled and sealed into 2 ml glass ampules while in an inert gas atmosphere. The ampules are collected and autoclave for final sterilization at 240° F. and 20 to 30 pounds pressure for about 30 minutes. Following the sterilization cycle, the ampules with Product R are cooled and washed.

All quantities are subject to plus or minus 2.5% variation for pH, volume, and analytical adjustments.

### Method II For Preparing Product R

Suspend about 35.0 g of casein, about 17.1 g of beef peptone, about 22.0 g of nucleic acid (RNA), about 3.25 g bovine serum albumin in about 2.5 liters of water for injection USP at about 3 to 7° C. in a suitable container and gently stir until all the ingredients have been properly wet. Slowly add while stirring about 11.75 ml of hydrochloric acid (reagent grade ACS) and continue stirring until hydrochloric acid is completely dissolved. Autoclave at about 9 lbs pressure and 200-230° F. for a period of time until RNA is completely digested, for example, about 4 hours. At the end of the period, the autoclave is stopped and the reaction flask and contents are permitted to slowly cool to ambient temperature. Then cool for at least six hours at about 3-8° C. The resulting solution is filtered through 2 micron and 0.45 micron filters using inert gas such as nitrogen or argon at low pressure (1-6 psi). In a similar manner the solution is filtered again through 0.2 micron pyrogen retention filters. The resulting filtrate is sampled and assayed for total nitrogen. A calculation is then performed to determine the quantity of cooled water for injection to be added to the filtrate to yield a diluted filtrate with a nitrogen content between about 165-210 mg/mil, the final volume is approximately 5 liters. The pH is then adjusted with either concentrated HCL (reagent grade ACS) or 35% (w/v) of NaOH to about 7.3-7.6 range. The diluted solution is then filtered again through 0.2 micron filters with inert gas at low pressure. The final filtrate is then filled and sealed into 2 ml glass ampules while in an inert gas atmosphere. The ampules are collected and autoclave for final sterilization at 240° F. and 20 to 30 pounds pressure for about 30 minutes. Following the sterilization cycle, the ampules with Product R are cooled and washed.

All quantities are subject to plus or minus 2.5% variation for pH, volume, and analytical adjustments.

The following description represents the preferred embodiment of the present invention. Other methods apparent to a person of ordinary skill in the art may also be used to achieve the same objects as described below.

Cell culture: H9 T lymphoma or U937 promonocyte cells (ATCC, Rockville, Md.) are cultured in any suitable medium and under any conditions suitable for the proliferation of those cells. Preferably, H9 and U937 cells are cultured in RPMI 1640 medium (GIBCO-BRL, Gaithersburg, Md.) supplemented with 20% fetal bovine serum (HyClone, Logan, Utah), Lglutamine at 2 mM and penicillin/streptomycin(GIBCO-BRI) at 100 U/ml at 37° C. with 5% CO₂ in the atmosphere. The initial culture is screened for potential contamination of mycoplasma using a commercial kit (ATCC) and for HIV-1 by RT-PCR (see below).

Electroporation of cells: To introduce Product R into H9 or U937 cells, the cells from the above cultures are harvested at the exponential phase by centrifugation. Preferably, the cells are centrifuged at 1200 r.p.m at 40° C. for 10 minutes. Then the liquid portion of the culture is removed, leaving the cell pellets (about 4×10⁶) at the bottom of the centrifuge tubes. The cell pellets are resuspended in 20 ml of a suitable medium, preferably serum-free RPMI 1640 medium and then centrifuged again. After the second centrifugation, the medium is completely removed. The cells are resuspended in various concentrations of Product R diluted with cold serum-free RPMI 1640 medium or other suitable medium. The resuspended cells are then transferred into electroporation cuvettes (for example, cuvettes of 4 mm gap, BTX), about 400 μl per cuvette. The electroporation is performed at a voltage of about 150 V, preferably, using an electroporator from BTX (ECM 395, BTX, San Diego, Calif.) or from any other manufacturer of a like machine. After the electroporation, the cells are transferred into a culture flask that contains 15 milliliters of complete medium in each, and cultured under standard conditions as would be apparent to a person of ordinary skill in the art, for example at 37° C.-5% CO₂ for about 14 to 18 hours. To evaluate the effects of Product R on the viability of the cells and on nonspecific inhibitions of gene expression, control cells, i.e. the electroporated H9 or U937 cells in the absence of Product R, are employed. It has been found, Product R does not significantly affect the viability of these cells. Previous experiments have shown that Product R does not inhibit the expression of IL-2. ). J. Investig. Med 1996; 44:347-351. Thus, the level of the IL-2 expression of the cells may be used to determine nonspecific inhibitions of gene expressions. It has also been found that the IL-2 expression of the control cells is not significantly changed, indicating that little nonspecific inhibition occurs.

Preparation of total RNA: The electroporated H9 or U937 cells are harvested by centrifugation. Preferably, the cells are centrifuged at about 1200 r.p.m at 4° C. for about 10 minutes. After the complete removal of the medium, the cell pellets are used to prepare the total RNA by any standard method known to a person of ordinary skill in the art, preferably using Ultraspec-II RNA kit (Biotecx, Houston, Tex.) following the manufacturer's instructions. The amount of RNA in each preparation is determined by way of spectrophotometry at the absorbance of 260 nm and the quality of the RNA preparation is evaluated by a ratio of OD.260 nm to OD. 280 nm. An RNA preparation having an OD260/OD280 ratio of 1.6-1.8 is suitable for use in the synthesis of the first strand of a cDNA.

Reverse transcription- polymerase chain reaction (RT-PCR): After the preparation of the total RNA, the first strand of a cDNA is synthesized according to any well known protocol such as, for example, the protocol described by Promega, Madison, Wis., as modified in accordance with the ensuing description. Briefly, the total RNA, preferably about 800 ng, is heated in the presence of an oligo(dT) primer, preferably about 0.5 μg, at about 65° C. for about 10 minutes and then at room temperature for about another 2 minutes. Then the total RNA is mixed with AMV reverse transcriptase buffer, dNTPs at 1 mM for each nucleotide, MgSO₄ at 5 mM and AMV reverse transcriptase (about 20 U) in a total reaction volume of 20 μl. The reverse transcription is carried out at 42° C. for 60 minutes. Two microliters of the resulting first strand of a cDNA are taken for PCR according to the Promega instruction. The optimal concentration of Mg⁺⁺ for each oligonucleotide primer is determined in a preliminary experiment. Primers for the mRNAs of HIV coreceptors CCR3(1), CCR5(2), CXCR4(3), or GAPDH (4), or HIV-1 (5) are synthesized by Genosys, Woodland, Tex. or other companies and used in the PCR at 1 μM each. The reaction is carried out at 95° C. for 1 minute, then at 55° C. for 2 minutes and then at 72° C. for 2 minutes to complete 35 cycles, and then at the end of the cycles the reaction continues at 72° C. for another 10 minutes. The PCR products of 539 bp, 280 bp, 381 bp, 195 bp and 250 bp represent CCR3, CCR5, CXCR4, GAPDH and HIV-1 expressions, respectively, as known by a person of ordinary skill in the art.

Evaluation of PCR products: the quantity of each PCR product is measured by any well known technique, preferably electrophoresis, although other known evaluation techniques may be employed. Preferably ten microliters of each PCR product are loaded in a 1.5% agarose gel with ethidium bromide at 0.2 μg/ml both in the gel and in the running buffer (TAE, pH 7.5). A 100-bp ladder molecular weight standard such as one made by Promega is used as molecular weight references. The amplicons of the PCR are visualized under UV light and photographed to yield the measurement.

The present invention provides a basis for the treatment or the prevention of HIV infections by inhibiting HIV coreceptor CCR5 using Product R. It may be used to prevent or limit HIV infection by administration immediately after the patient's exposure to HIV, for example, within the first 24 to 48 hours after exposure. Such inhibition may be achieved by parenterally administering Product R to patients having HIV infections a suitable effective CCR5 inhibition dose of Product R in the range of from about 2.5 microliters to about 40 microliters per kilogram of body weight per day, preferably in the range of about 5 microliters to about 25 microliters per kilogram of body weight per day. Most preferably Product R is administered in an amount of about 7.5 microliters per kilogram of body weight per day. The desired dose may be administered as two, three or more sub-doses at appropriate intervals, generally equally spread in time, throughout the day. Preferably, the full daily dose is administered in one administration.

Product R may be administered by any suitable injection route including, but not limited to intravenously, intraperitoneally, subcutaneously, intramuscularly, and intradermally, etc. The presently preferred route of administration is intramuscularly. It will be appreciated that the preferred route may vary with, for example, the condition and age of the recipient.

Since Product R has proven to be toxicity free, the period of administration may be varied depending on the patient condition, such as from a few days to a year or infinite time.

The following examples serve as illustrations to the present invention are not intended to limit the scope of the claims defining the present invention.

### EXAMPLE 1

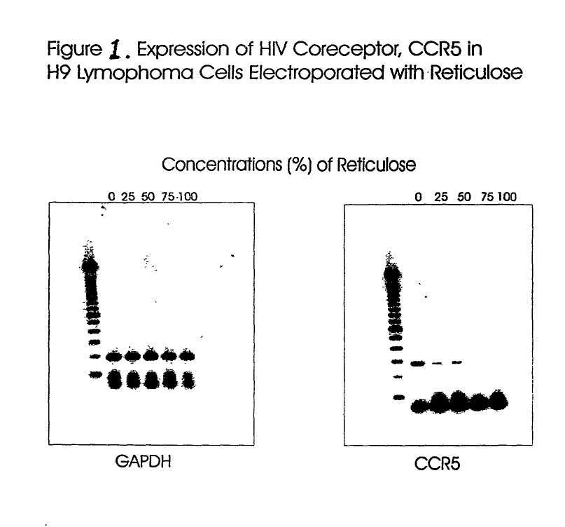

As shown in FIG. 1, H9 T lymphoma cells are treated according to the methods described above. Particularly, the H9 cells were electroporated in Product R at the concentrations indicated in FIG. 1, i.e. 0%, 25%, 50%, 75% and 100%. After 16 hours of culturing, a dose-responsive reduction of the expression of CCR5 was detected by RT-PCR in Product R-treated cells (the panel on the right). In contrast, such reduction is not observed from the internal control, the GAPDH gene expression (the panel on the left), demonstrating the specific effect of Product R on the expression of the CCR5 gene. The Product R significantly reduces CCR5 expression at a concentration of 75% according to visual observation.

### EXAMPLE 2

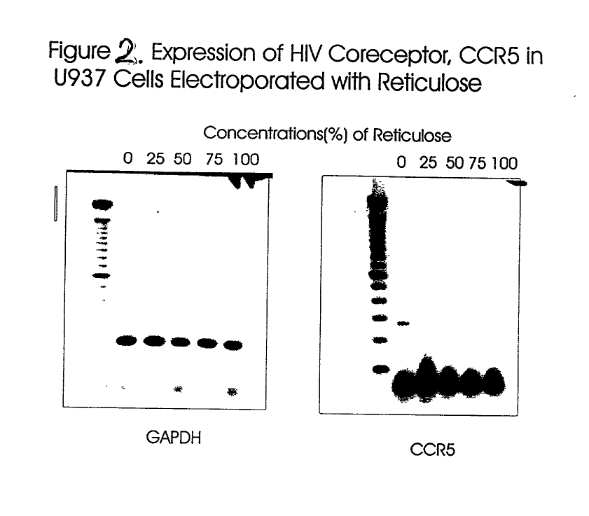

FIG. 2 shows a dose-responsive reduction of the expression of CCR5 by Product R at the concentrations of 0%, 25%, 50%, 75% and 100% in U937 cells. The U937 cells were treated as those in EXAMPLE 1. A significant reduction of the CCR5 expression can be visually observed as the Product R concentration is increased (panel on the right). In contrast, such reduction is not observed from the internal control, the GAPDH expression (panel on the left). Compared with FIG. 1 of EXAMPLE 1, U937 cells appears to be more sensitive to Product R than H9 cells, because CCR5 PCR product in U937 cells cannot be visually observed at the concentration of 25%, but can be observed in H9 cells.

The invention is not limited to the embodiments described above which are presented as examples only but can be modified in various ways within the scope of protection defined by the appended patent claims.

We claim:

1. A method for determining down-regulation of gene expression of a human immunodeficiency virus (HIV) coreceptor, comprising the steps of:
a. culturing H9 T lymphoma or U937 promonocyte cells;
b. electroporating said H9 or said U937 cells to introduce Product R into said H9 or said U937 cells;
c. culturing said electroporated H9 or said U937 cells;
d. preparing total RNA from said H9 or said U937 cells after the step c;
e. reverse-transcribing the mRNA of said HIV coreceptor by a reverse transcription-polymerase chain reaction (RT-PCR); and
f. measuring said RT-PCR product.

2. The method of claim 1, wherein said HIV coreceptor is CCR5.

3. The method of claim 1, wherein said step f further comprises the step of electrophorizing said RT-PCR.

4. The method of claim 1, wherein said electroporated H9 or U937 cells are cultured for about 14 hours to about 18 hours.

5. A method for reducing the amount of HIV coreceptor CCR5 in a patient having HIV infection, comprising parenterally administering an effective HIV coreceptor CCR5 reduction amount of Product R to said patient.

6. A method for preventing a patient from being infected by HIV, comprising parenterally administering an effective HIV coreceptor CCR5 reduction amount of Product R to said patient.
